(19) United States
(12) Patent Application Publication
(10) Pub. No.: US 2020/0337971 A1
(43) Pub. Date: Oct. 29, 2020

(54) ANTI-MICROBIAL SOAP

(71) Applicant: SANIT TECHNOLOGIES LLC, Sarasota, FL (US)

(72) Inventor: Joseph GIOVANNIELLO, Wayne, NJ (US)

(21) Appl. No.: 16/962,124

(22) PCT Filed: Jan. 15, 2019
(86) PCT No.: PCT/US2019/013582

§ 371 Date: Jul. 14, 2020

Related U.S. Application Data
(60) Provisional application No. 62/617,346, filed on Jan. 15, 2018.

(51) Int. Cl. A61K 8/44; A61K 8/41; A61K 8/34; A61K 8/92; A61Q 17/00; A61Q 19/10

(52) U.S. Cl. CPC A61K 8/44; A61K 8/416; A61K 8/34; A61Q 19/10; A61K 8/922; A61K 8/927; A61Q 17/005; A61K 8/345

(57) ABSTRACT

The instant application relates to an antimicrobial soap. The soap may comprise water, ethylenediaminetraacetic acid, cocamidopropyl betaine, at least one fatty additive, at least one quaternary amine, and benzyl alcohol.

### CROSS REFERENCE TO RELATED APPLICATIONS

This application claims priority to U.S. Provisional Application No. 62/617,346, filed Jan. 15, 2018, the entire contents of which are incorporated herein by reference.

### BACKGROUND

Antibacterial soaps have historically been popular with consumers. Unfortunately, the Food and Drug Administration discovered that many soaps identified as “antibacterial” soaps offered no additional protection or antimicrobial properties beyond those offered by regular soap and water. Additionally, ingredients historically used in antibacterial soaps raised health concerns related to long term exposure to those ingredients. The FDA has banned nineteen (19) ingredients commonly and historically used in antibacterial soap, including triclosan, which is found in approximately 75% of anti-bacterial liquid soaps in America. Further concerns with prior art antibacterial soaps was that these soaps could lead to antibiotic resistance. Additionally, some soaps include alcohol, such as isopropanol and/or ethyl alcohol, which are flammable, and can lead to dry skin and related conditions. In light of this, there is a need for a soap that comprises safe ingredients and can kill antimicrobial agents without increasing the threat from superbugs.

### SUMMARY

One embodiment of the soaps described herein relates to an antimicrobial soap. The anti-microbial soap comprises water, ethylenediaminetraacetic acid, cocamidopropyl betaine, at least one fatty additive, at least one quaternary amine, and benzyl alcohol.

Another embodiment relates to an antimicrobial soap. The soap comprises 80.9+/−1.5% water, 0.40+/−1.5% ethylenediaminetraacetic acid, 15+/−1.5% cocoamidopropyl betaine, 1.0+/−1.5% fatty additive, and 2.5+/−1.5% benzyl alcohol. The water, ethylenediaminetraacetic acid, cocamidopropyl betaine, fatty additive, quaternary amine, and benzyl alcohol comprise particles have diameters less than or equal to 1 micron.

Another embodiment is directed to method of making an antimicrobial soap. Water and ethylenediaminetraacetic acid are mixed to form a first mixture. At least one quarternary amine, cocomidopropyl betaide, at least one fatty additive, and benzyl alcohol are added to the first mixture. The quarternary amine, cocomidopropyl betaide, fatty additive, and benzyl alcohol are mixed with the first mixture to form a blended mixture. The blended mixture is stabilized to for a soap.

### BRIEF DESCRIPTION OF THE DRAWINGS

FIG. 1 depicts a system that may be used with one embodiment of the instant methods.

### DETAILED DESCRIPTION

The compositions herein define anti-microbial soaps and methods for making the same. The anti-microbial soaps described herein do not use banned substances. Furthermore, the ingredients utilized in these soaps have shown effectiveness in killing antimicrobial agents.

The antimicrobial soap may comprise water. Preferably, the water is purified water. Examples of purified water include water that has been treated by filtration, demineralization, sedimentation, ultraviolet oxidation, desalination, boiling, distillation, dissolved air flotation, electrodialysis, activated carbon adsorption, reverse osmosis, and/or deionization. More preferably, the water is deionized water, and most preferably, the water is ultra-pure reverse osmosis dual filtered water.

The composition may comprise ethylenediaminetraacetic acid (EDTA). Preferably, EDTA is present in the composition as an EDTA salt. Examples of EDTA salts that may be used include tetrasodium EDTA, calcium disodium EDTA, and disodium EDTA. Preferably, the composition comprises EDTA disodium. EDTA disodium may be purchased commercially from vendors such as Dissolvine NA2-S. In the alternative, the composition may include citrate.

The composition may further comprise a fatty additive. The fatty additive may be a glycerol or a glycerol substitute. As used herein, glycerol, glycerin, and glycerine are used interchangeably.

In a preferred embodiment, the fatty additive is glycerol. The glycerol may be synthetic or derived from natural sources, such as animal fats, vegetable fats, or combinations thereof. Examples of animal sources for glycerol include fatty acids in tallow from beef, pork, and/or sheep and fatty acids found in dairy. Examples of vegetable sources of glycerol include soy beans, coconut oil, nutmeg, palm oil, palm kernel oil, and sunflower oil. Examples of synthetic glycerols include those made from petroleum and propylene. Preferably, the composition uses a 99% glycerine product. One exemplar glycerine product that may be utilized with the compositions described herein is the 99% glycerine product available from ACME-HARDESTY.

The glycerol may substituted, or used in conjunction, with other natural fat products. Examples of fatty additives that are glycerol substitutes, and may be used as described herein, include sweet almond oil, hazelnut oil, olive oil, coconut oil, canola oil, rice bran oil, apricot kernel oil, castor oil, chia seed oil, grapeseed oil, jojoba oil, hemp seed oil, meadowfoam oil, peanut oil, safflower oil, sunflower oil, tamanu oil, pumpkinseed oil, neem oil, coconut de crème, palm kernel flakes, beeswax, tallow, cocoa butter, avocado oil or butter, shea butter, mango butter, coffee butter, and combinations thereof.

The composition includes at least one quaternary amine compound. Preferably, the quaternary amine compound contains at least one long alkyl chain, and most preferably, the alkyl chain contains from 8 to 18 carbon atoms. Examples of quaternary amine compounds that could be utilized include benzalkonium chloride, stearalkonium chloride, cetylpyridine chloride, benzethonium chloride, methylbenzethonium chloride, cetalkonium chloride, cetylpyrindinium chloride, cetrimonium, cetrimide, dofanium chloride, tetraethylammonium bromide, alkyldimethylbenzylammonium chloride, dialkyldimethylammonium chloride, didecyldimethylammonium chloride, dioctyl dimethyl ammonium chloride, dodecyl dimethyl ammonium chloride, octyl decyl dimethyl ammonium chloride, alkyl (50% C₁₄, 40% C₁₂, and 10% C₁₆) dimethylbenzyl ammonium chloride, N,N-didecyl-N-methyl-poly(oxyethyl) ammonium propionate, quaternium-15, polyquaternium-9, domiphen bromide, and combinations thereof. Preferably, the quaternary amine is a benzalkoniumm chloride.

In one embodiment, the quaternary amine compound may comprise an aqueous quaternary amine compound of benzalkonium chloride. For example, the aqueous quaternary amine may be the product sold under the brand name MICROCARE QT from THOR.

In some embodiments, the soap composition further includes benzyl alcohol. Benzyl alcohol may be included isolated or as part of other formulas. For example, inclusion of various aqueous quaternary amine compounds of benzalkonium chloride may include benzyl alcohol (e.g. MICROCARE CTC and MICROCARE SBB) and can be used with the instant compositions.

The soap composition may additionally include cocoamidopropyl betaine. The cocoamidopropyl betaine may be commercially obtained from ACME-HARDESTY. The cocoamidopropyl betaine may be commercially obtained from StarChem (StarSurf™ CAPB).

Preferably, the particles of the components of the sanitizer have a particle diameter that is less than or equal to 1 micron. The diameter may be greater than or equal to 0.001 micron in some embodiments. Reducing the particle size allows for component particles to couple, resulting in improved antimicrobial properties, to include longer lasting protection from microbial agents.

One preferred embodiment of the composition described herein contains the ingredients as shown in Table 1 below:

TABLE 1
Components of one embodiment of the antimicrobial soap
composition. Measurements are percent by weight.

```
80.9% +/− 1.5% water
0.40% +/− 1.5% EDTA
0.20% +/− 1.5% quaternary amine compound
  15% +/− 1.5% cocoamidopropyl betaine
  1.0% +/− 1.5% glycerine
  2.5% +/− 1.5% benzyl alcohol
```

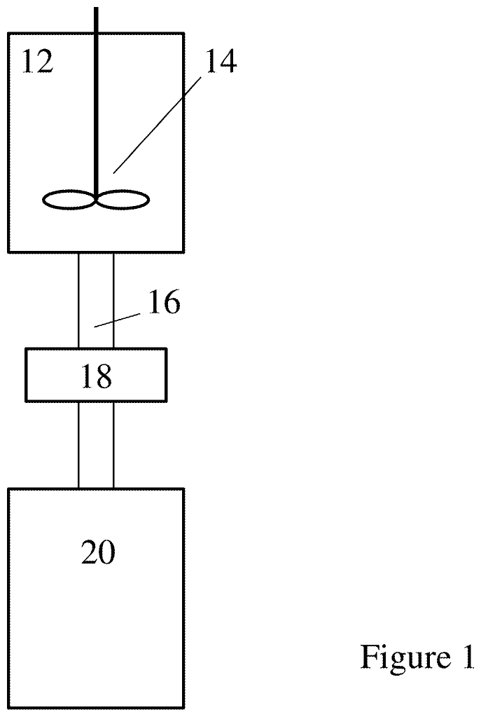

FIG. 1 shows a system that may be used with one embodiment of the instant methods. Blending vessel 12 and mixer 14 are provided. Blending vessel 12 may be a separate tank into which mixer 14 is placed, or blending vessel 12 and mixer 14 may be attached as a single unit. Blending vessel 12 may be any vessel capable of holding the Mixer 14 may be any mixer capable of creating a vortex while mixing, and preferably any mixer capable of reducing the size of the particles being mixed to particles with a diameter that is less than or equal to 1 micron. By way of example, blending vessel 12 may be a tank and mixer 14 may be a 25 horsepower Micro Sonic grinder mixer.

Amounts of each ingredient (see Table 1) may be calculated. Blending vessel 12 may be measured to determine how much final product it may hold. Alternatively, any amount of product that would fit within blending vessel 12 may be used.

In a preferred embodiment, most of the water is added to blending vessel 12. For example, if water is to comprise 80.9% of the final product, water amounting to 80% of the final product may be added, with the 0.9% to be added later as described herein. Alternatively, the entirety of the water component may be added initially.

EDTA is added to the blending vessel 12. After EDTA is added to the blending vessel 12, mixer 14 mixes the water and EDTA. Preferably, mixing lasts for at least three minutes.

After mixing the EDTA with the water, the quaternary amine, cocomidopropyl betaide, glycerine, and benzyl alcohol are added to blending vessel 12. These components may be added in any order, separately or at the same time as another of these components. The components may be added at any position in the tank. Preferably, they are added by pouring the components into the top, center of the blending vessel.

Mixer 14 mixes the contents of blending vessel 12. The length of mixing time depends on the amount of soap being made. For an approximately 2,000 lb batch of soap, mixer 14 mixes the contents of pre-mix blending vessel for at least 10 minutes and, preferably, at least 15 minutes.

After mixing, the contents of blending vessel 12 are stabilized. Stabilizing involves allowing the contents of blending vessel 12 to sit in the vessel for at least sixty (60) minutes.

Any remaining water that was not added initially is added to vessel 12. This second water addition could be made before or after stabilization or any time during stabilization.

After stabilization, the contents of blending vessel 12 may be filtered. By way of an example, blending vessel 12 may further include a hose 16 or other outlet. A filtration unit 18 may be attached to hose 16. The filtration unit may be a filtration unit bag, and, preferably, is a 150 Mesh filtration unit bag. The contents of blending vessel 12 are directed through hose 16 and through filtration unit 18 to create a filtered soap. Preferably, the contents of blending vessel 12 are directed through hose 16 and through filtration unit 18 via gravity transfer. The filtered soap is then transferred into tank 20.

Although the present composition has been shown and described in considerable detail with respect to only a few/particular exemplary embodiments thereof, it should be understood by those skilled in the art that it is not intended to limit the composition to the embodiments since various modifications, omissions, and additions may be made to the disclosed embodiments without materially departing from the novel teachings and advantages of the composition, particularly in light of the foregoing teachings.

1. An antimicrobial soap, comprising water, ethylenediaminetraacetic acid, cocamidopropyl betaine, fatty additive, quaternary amine, and benzyl alcohol.

2. The antimicrobial soap of claim 1, wherein the fatty additive is glycerol.

3. The antimicrobial soap of claim 1, wherein the fatty additive is a glycerol substitute.

4. The antimicrobial soap of claim 3, wherein the glycerol substitute is sweet almond oil, hazelnut oil, olive oil, coconut oil, canola oil, rice bran oil, apricot kernel oil, castor oil, chia seed oil, grapeseed oil, jojoba oil, hemp seed oil, meadowfoam oil, peanut oil, safflower oil, sunflower oil, tamanu oil, pumpkinseed oil, neem oil, coconut de crème, palm kernel flakes, beeswax, tallow, cocoa butter, avocado oil or butter, shea butter, mango butter, coffee butter, or combinations thereof.

5. The antimicrobial soap of claim 1, wherein the quaternary amine is benzalkonium chloride.

6. The antimicrobial soap of claim 1, wherein the ethylenediaminetraacetic acid is ethylenediaminetraacetic acid disodium.

7. The antimicrobial soap of claim 1, wherein the water, ethylenediaminetraacetic acid, cocamidopropyl betaine, fatty additive, quaternary amine, and benzyl alcohol comprise particles with diameters less than or equal to 1 micron.

8. The antimicrobial soap of claim 1, wherein the water comprises 80.9+/−1.5% of the soap.

9. The antimicrobial soap of claim 1, wherein the ethylenediaminetraacetic acid comprises 0.40+/−1.5% of the soap.

10. The antimicrobial soap of claim 1, wherein the quaternary amine comprises 0.2+/−1.5% of the soap.

11. The antimicrobial soap of claim 1, wherein the cocoamidopropyl betaine comprises 15+/−1.5% of the soap.

12. The antimicrobial soap of claim 1, wherein the fatty additive comprises 1.0+/−1.5% of the soap.

13. The antimicrobial soap of claim 1, wherein the benzyl alcohol comprises 2.5+/−1.5% of the soap.

14. An antimicrobial soap comprising 80.9+/−1.5% water, 0.40+/−1.5% ethylenediaminetraacetic acid, 15+/−1.5% cocoamidopropyl betaine, 1.0+/−1.5% fatty additive, and 2.5+/−1.5% benzyl alcohol, wherein the water, ethylenediaminetraacetic acid, cocamidopropyl betaine, fatty additive, quaternary amine, and benzyl alcohol comprise particles with diameters less than or equal to 1 micron.

15. The antimicrobial soap of claim 14, wherein the fatty additive is glycerol.

16. The antimicrobial soap of claim 14, wherein the fatty additive is a glycerol substitute.

17. A method of making an antimicrobial soap, the method comprising:
mixing water and ethylenediaminetraacetic acid to form a first mixture;
adding at least one quarternary amine, cocomidopropyl betaide, a fatty additive, and benzyl alcohol to the first mixture;
mixing the at least one quarternary amine, cocomidopropyl betaide, a fatty additive, and benzyl alcohol with the first mixture to form a blended mixture; and
stabilizing the blended mixture to form a soap.

18. The method of claim 17, wherein stabilizing the blending mixture comprises settling the blended mixture.

19. The method of claim 17, further comprising filtering the blended mixture.

20. The method of claim 17, wherein water is added to the blended mixture while the blended mixture is stabilized.

21. The method of claim 17, wherein water is added to the blended mixture before the blended mixture is stabilized.

22. The method of claim 17, wherein the water is added after the blended mixture is stabilized.

23. The method of claim 17, wherein the water comprises 80.9+/−1.5% of the soap.

24. The method of claim 17, wherein the ethylenediaminetraacetic acid comprises 0.40+/−1.5% of the soap.

25. The method of claim 17, wherein the quaternary amine comprises 0.2+/−1.5% of the soap.

26. The antimicrobial soap of claim 1, wherein the cocoamidopropyl betaine comprises 15+/−1.5% of the soap.

27. The method of claim 17, wherein the fatty additive comprises 1.0+/−1.5% of the soap.

28. The method of claim 17, wherein the benzyl alcohol comprises 2.5+/−1.5% of the soap.

29. The method of claim 17, wherein mixing reduces the size of the particles of water, ethylenediaminetraacetic acid, cocamidopropyl betaine, fatty additive, quaternary amine, and benzyl alcohol to particles with diameters less than or equal to 1 micron.
